(19) United States
(12) Patent Application Publication
(10) Pub. No.: US 2026/0083495 A1
(43) Pub. Date: Mar. 26, 2026

(54) BIPOLAR RF ACCESS DEVICE WITH SLIDING AUTOMATIC SHUTOFF CUTTING ZONE

(71) Applicant: Boston Scientific Scimed, Inc., Maple Grove, MN (US)

(72) Inventor: Robb Morse Gavalis, Westborough, MA (US)

(73) Assignee: Boston Scientific Scimed, Inc., Maple Grove, MN (US)

(21) Appl. No.: 19/336,823

(22) Filed: Sep. 23, 2025

Related U.S. Application Data
(60) Provisional application No. 63/697,694, filed on Sep. 23, 2024.

(51) Int. Cl. A61B 18/14; A61B 18/00; A61B 18/16; A61M 25/06

(52) U.S. Cl. CPC A61B 18/1487; A61B 18/1492; A61B 2018/00077; A61B 2018/00083; A61B 2018/00577; A61B 2018/00601; A61B 2018/00708; A61B 2018/00982; A61B 2018/162; A61M 2025/0681

(57) ABSTRACT

An electrosurgical ablation device and associated methods for treating patient tissue are disclosed herein. Example electrosurgical ablation devices of the present disclosure may include an outer sheath having a proximal end, a distal end, a lumen extending therebetween, and an active electrode disposed proximate to the distal end of the outer sheath. Example electrosurgical ablation devices of the present disclosure may further include an access cannula slidably disposed within the outer sheath and having a body composed of an electrically conductive material including an insulating material applied to one or more portions of the body to form one or more shutoff sections and one or more cutting sections. The one or more cutting sections and the active electrode may be adapted to couple to a generator and form a conductive path to ablate tissue.

### CROSS-REFERENCE TO RELATED APPLICATIONS

This application claims the benefit of priority of U.S. Provisional Application No. 63/697,694 filed Sep. 23, 2024, the entire disclosure of which is hereby incorporated by reference.

### TECHNICAL FIELD

The present disclosure relates generally to medical devices and methods. More particularly, the present disclosure relates to RF delivery devices utilized in various treatments and interventions.

### BACKGROUND

Transmural access into the biliary tree or deeper into the gastrointestinal (GI) tract are procedures that are non-surgical and non-percutaneous options that can be implemented when standard endoscopic retrograde cholangiopancreatography (ERCP) fails. Although ERCP is a preferred method to diagnose and treat problems of the biliary or pancreatic ductal systems. Unfortunately, ERCP is not always an option or successful in difficult anatomical cases or with challenging disease states.

Transmural drainage is a preferred option for such challenging cases. For example, choledochodudenostomy (CDS) and hepaticogastrostomy (HGS) procedures can be employed where ERCP is unsuitable or unsuccessful. Further, endoscopic ultrasound-directed transgastric ERCP (EDGE) procedures are also increasing in utilization as the use of lumen opposing metal stents have increased.

The above procedures are typically performed under endoscopic ultrasound (EUS) to visualize the target anatomy through the wall of the GI tract. During these procedures, a new tract is made by penetrating through tissue and dilating the new tract to create an artificial access point to the target anatomy. One of the methods to dilate the tract is cutting, for example, using radiofrequency (RF) energy. This is often done using a cystotome, or similar access system. Many of these cutting systems utilize a monopolar RF electrode with a grounding pad applied on the patient that acts as the return electrode. Although these systems function well, the cutting location is solely controlled by advancing the cutting electrode itself. Some devices include very long cutting electrodes (e.g., five feet or longer). As this is advanced through an endoscope and visualized under EUS, controlling the precision of the cutting can be challenging.

As such, there is a need for cystotome or other like cutting and dilating systems that provide for greater control, precision, and accuracy.

### BRIEF SUMMARY

The present disclosure provides improved methods, devices and alternative uses for controlled energy delivery to a desired treatment site of a patient. More particularly, the present disclosure provides improved methods, devices and alternative uses for controlled bipolar energy delivery to a desired treatment site of a patient.

Accordingly, the present disclosure provides an electrosurgical ablation device for treating patient tissue. The electrosurgical ablation device of this and other examples may include an outer sheath including a proximal end, a distal end, a lumen extending therebetween, and an active electrode disposed proximate to the distal end of the outer sheath. The electrosurgical ablation device of this and other examples may also include an access cannula having a body composed of an electrically conductive material including an insulating material applied to one or more portions of the body to form one or more shutoff sections and one or more cutting sections. The access cannula may be adapted to be slidably disposed within the outer sheath, and the one or more cutting sections and the active electrode may be adapted to couple to a generator and form a conductive path to ablate tissue.

Alternatively, or additionally to any of the examples described herein, the conductive path may be maintained as the access cannula is slidably moved through the outer sheath.

Alternatively, or additionally to any of the examples described herein, the access cannula may be composed of two or more electrically conductive materials.

Alternatively, or additionally to any of the examples described herein, the one or more shutoff sections may be composed of two or more insulating materials.

Alternatively, or additionally to any of the examples described herein, the one or more shutoff sections may include a dielectric material.

Alternatively, or additionally to any of the examples described herein, the outer sheath may further include a distal insulating tip member disposed proximate the active electrode.

Alternatively, or additionally to any of the examples described herein, the active electrode may be one of a ring electrode, an annular electrode, a cutting ring electrode, a cutting annular electrode, a conical electrode, a frustoconical electrode, and/or an electrode including a plurality of projections.

Alternatively, or additionally to any of the examples described herein, the access cannula may be a guidewire or a tissue dilator.

Alternatively, or additionally to any of the examples described herein, the insulating material applied to the one or more portions of the body may be applied in the form of a jacket, a coating, or a covering.

Alternatively, or additionally to any of the examples described herein, the insulating material applied to the one or more portions of the body may be applied in the form of a partial jacket, a partial coating, or a partial covering.

Alternatively, or additionally to any of the examples described herein, one or both one or more cutting sections and the one or more shutoff sections may be sized and dimensioned in relation to a size of an intended treatment site.

Alternatively, or additionally to any of the examples described herein, the insulating material applied to the one or more portions of the body may be applied to the body in a pattern selected from the group including: a repeatably intermittent pattern, an intermittent pattern of varying sectional length, a dotted pattern, a relief pattern, a zig-zag pattern, a spiral pattern, a sinusoidal pattern, a hyperbolic pattern, and a mesh pattern.

Alternatively, or additionally to any of the examples described herein, the one or more shutoff sections may interrupt the current pathway between the active electrode and the one or more cutting sections.

Alternatively, or additionally to any of the examples described herein, the conductive path may include an energy activation region, and a boundary of the energy activation region may be set at a preset distance from the active electrode.

Alternatively, or additionally to any of the examples described herein, the preset distance may be a distance between 0.005 mm and 10 cm.

Further contemplated by the present disclosure are methods for ablating tissue using an electrosurgical ablation device as disclosed herein. An example method for ablating tissue may include providing an electrosurgical ablation device including an outer sheath having a proximal end, a distal end, a lumen extending therebetween, and an active electrode disposed proximate to the distal end of the outer sheath. The electrosurgical ablation device may further include an access cannula having a body composed of an electrically conductive material including an insulating material applied to one or more portions of the body to form one or more shutoff sections and one or more cutting sections. The method of this and other examples may include positioning an endoscope within a first body lumen of a patient adjacent to a desired treatment site. The method of this and other examples may further include advancing the electrosurgical ablation device through the endoscope and into the desired area of treatment such that at least one cutting section may be placed within the desired area of treatment. Thereafter, the method of this and other examples may further include activating an electrosurgical generator to deliver energy to the electrosurgical ablation device and applying energy to the treatment site through the active electrode via the electrosurgical generator to locally ablate tissue when the active electrode passes over the cutting section of the access cannula. The method of this and other examples may further include automatically ceasing application of energy to the treatment site when the active electrode passes over the one or more shutoff sections of the access cannula.

### BRIEF DESCRIPTION OF THE SEVERAL VIEWS OF THE DRAWINGS

The disclosure may be more completely understood in consideration of the following detailed description in connection with the accompanying drawings, in which:

FIG. 1A illustrates a deployed electrosurgical ablation device accompanied with an endoscopic ultrasound (EUS) access system and related components for accessing a tissue treatment site.

FIG. 1B is a diagram depicting the interconnectedness of various elements of examples and embodiments the present disclosure.

FIG. 2 illustrates a side view of an electrosurgical ablation device in accordance with examples and embodiments of the present disclosure.

FIG. 3 illustrates an example of a deployed electrosurgical ablation device within the GI tract of a patient or subject.

FIG. 4 illustrates a conventional dilation catheter of the prior art, which utilizes monopolar RF energy, and a grounding pad attached to the patient or subject.

FIG. 5 illustrates a device of the present disclosure which may include a slidably engaged bipolar electrode system.

FIG. 6 illustrates an example electrode configuration for the systems and devices disclosed herein.

FIG. 7 illustrates a modified EUS access device with controlled electrode distance within the bipolar cutting region.

### DETAILED DESCRIPTION

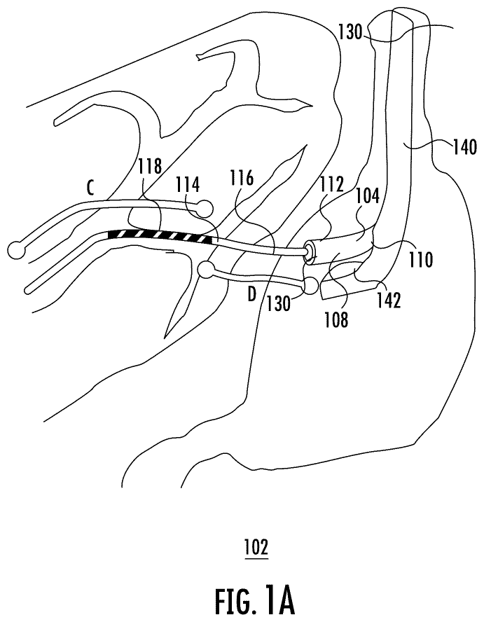

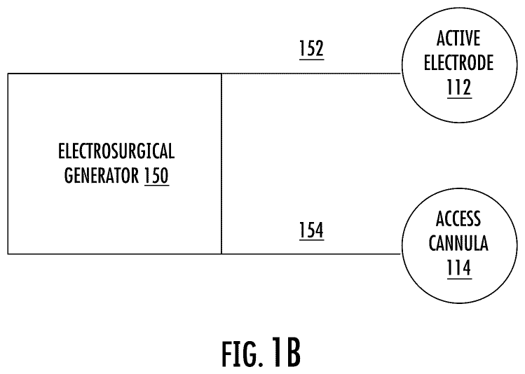

FIG. 1A illustrates an electrosurgical ablation device 102 extending out the distal end of an endoscope 140 positioned in a patient's stomach 120. The electrosurgical ablation device 102 can be inserted through a working channel of the endoscope 140 (e.g., during an endoscopic ultrasound (EUS) procedure, or the like) and used to access the biliary or pancreatic ductal systems of a patient. More particularly, and as shown in FIG. 1A, the electrosurgical ablation device 102 is used to gain access to the bile ducts 124 of the liver 122 from the stomach 120.

The electrosurgical ablation device 102 of this and other examples may include an outer sheath 104 having a lumen (not shown) and an access cannula 114 disposed within the lumen of the outer sheath 104. The access cannula 114 may be slidable within the lumen and/or slidable relative to the outer sheath 104. Alternatively, or additionally, the outer sheath 104 may be slidable about and/or along the access cannula 114 in one or more of a coaxial, concentric, telescoping, and/or nested relationship and/or may be slidable relative to the access cannula 114. The outer sheath 104 has an electrode 112 (e.g., active electrode) disposed proximate to the distal end 108 of the outer sheath 104. Electrode 112, and other associated and/or adjacent components may be disposed in a slidable arrangement with access cannula 114. In other words, electrode 112, and/or other associated and/or adjacent components may move relative to the access cannula 116 in ways including but not limited to translation, rotation, movement along an axis, and/or any combination or permutation of the aforementioned. The outer sheath 104 may include an insulating element 130 disposed proximate to the distal end 108 of the outer sheath 104, the active electrode 112, or both. As described herein, insulating element 130 may be disposed in a slidable arrangement with access cannula 114. In other words, insulating element 130 and/or other associated and/or adjacent components may move relative to the access cannula 114 in ways including but not limited to translation, rotation, movement along an axis, and/or any combination or permutation of the aforementioned.

Additionally, and as will be described further herein, insulating element 130 may govern a controlled ablation and/or cutting and/or emission distance between the electrode 112 from the access cannula 114, thereby aiding to form a customizable treatment range within and/or proximate a desired treatment area. In some non-limiting instances, the controlled ablation and/or cutting and/or emission distance between the electrode 112 and the access cannula 114 may be controlled and/or otherwise modified such that the controlled ablation and/or cutting and/or emission distance between the electrode 112 and the access cannula is a preset and/or preselected distance that is proximate, or at least substantially proximate to the electrode 112. In this and other examples, the preset and/or preselected distance may be a distance in the range of 0.1 mm to 2.5 mm or more. Alternatively, or additionally, the electrode 112 may emit energy suitable for controlled ablation and/or cutting and/or dilation at an emission distance ranging between 0.1 mm to 2.5 mm or more when the electrode 112 is within the cutting section 116 of access cannula 114. In other words, the electrode 112 is within the cutting section 116 of access cannula 114 in such non-limiting scenarios including: when the electrode 112 eclipses any portion of the cutting section 116 of access cannula 114 and/or when the electrode 112 is within a preset distance of the one or more insulated sections 118 of access cannula by any preset and/or preselected distance described herein, and/or when the electrode 112 is within a proximal distance of any portion of the cutting section 116 of access cannula, whereby a “proximal distance” may be any distance ranging from 0.1 mm to 2.5 mm. Alternatively or additionally, electrode 112 may emit energy in the energy-emission shape and/or geometry of an arc, in a linear formation, a curvilinear formation, a projecting concavity, a projecting convexity, a projecting sphere, a projecting bubble, and/or any combination and/or permutation of the aforementioned energy-emission shapes and/or geometries.

With some examples, the endoscope 140 can include an ultrasound transducer 142, which aids in locating the desired treatment site through ultrasonic imaging (e.g., EUS). However, other imaging modalities are contemplated for use with the disclosed elements described herein, including but not limited to computed tomography (CT), X-ray, magnetic resonance imaging (MRI), nuclear magnetic resonance imaging (nMRI), fluoroscopy, mammography, positron emission tomography (PET), or other like imaging modalities.

Access cannula 114 has at least one conductive portion 116 (also referred to as a cutting section 116) which thereby renders access cannula 114 a return electrode. Alternatively or additionally, and by virtue of access cannula 114 including at least one conductive portion (including but not limited to the immediately preceding example), access cannula 114 may be referred to as a ground electrode as the at least one conductive portion 116 offers the least resistance to the current pathway flowing through electrode 112. Access cannula may include at least one insulated portion 118 (also referred to as a shutoff section 118). One or more insulated portions 118 may be disposed adjacent to, about, and/or proximate one or more cutting sections 116. In yet other non-limiting examples, access cannula 114 can be formed from a conductive body with selectively insulated sections, thereby forming the cutting section 116 (e.g., where the conductive body is devoid of insulating material) and the shutoff section 118 (e.g., where the conductive body has insulating material applied).

As described herein, the access cannula 114 may be slidable within the lumen and/or slidable relative to the outer sheath 104. Alternatively, or additionally, the outer sheath 104 may be slidable about and/or along the access cannula 114 in one or more of a coaxial, concentric, telescoping, and/or nested relationship. In this and other examples, the access cannula 114 may be alternatively fixed within a portion of the outer sheath 104, within a portion of the outer sheath lumen, and/or held fixed proximate to and/or distal to the distal end of the outer sheath 104.

As described herein, the outer sheath 104 has an electrode 112 (e.g., active electrode) disposed proximate to the distal end 108 of the outer sheath 104. Electrode 112, and other associated and/or adjacent components may be disposed in a slidable and/or fixed arrangement with access cannula 114. In other words, electrode 112, and/or other associated and/or adjacent components may move relative to the access cannula 116 in ways including but not limited to translation, rotation, movement along an axis, and/or any combination or permutation of the aforementioned. Alternatively or additionally, electrode 112, and/or other associated and/or adjacent components may be held in fixed relation to the access cannula 116 in ways creating a controlled linear distance between the two while still allowing movement in other directions, including but not limited to a telescopic and/or nested connection, and/or other known connection and/or coupling techniques known in the art. The outer sheath 104 may include an insulating element 130 disposed proximate to the distal end 108 of the outer sheath 104, the active electrode 112, or both. As described herein, insulating element 130 may be disposed in a slidable and/or fixed arrangement with access cannula 116. In other words, insulating element 130 and/or other associated and/or adjacent components may move relative to the access cannula 116 in ways including but not limited to translation, rotation, movement along an axis, and/or any combination or permutation of the aforementioned. Alternatively or additionally, insulating element 130, and/or other associated and/or adjacent components may be held fixed relative to the access cannula 116 in ways including but not limited to an interference fit connection, a snap-fit connection, a telescopic and/or nested connection, and/or other known connection and/or coupling techniques known in the art.

Additionally, and as will be described further herein, insulating element 130 may govern a controlled ablation and/or cutting and/or emission distance between the electrode 112 from the access cannula 114, thereby aiding to form a controlled electrode pair that can deliver energy. The controlled distance between electrodes 112 and 116 may be held consistent even when the outer sheath 104 is moving relative to the access cannula 114, creating a customizable treatment range within and/or proximate a desired treatment area.

During operation, the cutting section 116 acts as a conductor to form a conductive path with the electrode 112 when tissue is pressed between the electrodes. As such, where the device 102 is coupled to an electrosurgical generator 150 (e.g., as shown by the diagram in FIG. 1B) an access path to the bile ducts 124 can be formed as electrode 112 passes over the cutting section 116 when energy is applied. It can be appreciated that the energy emitted from electrode 112 may be precisely controlled such that therapy may be exclusively executed in an energy emission region 145. The energy emission region 145 may be dictated by the structural and/or functional relationship of the active electrode 112 and the one or more cutting regions 116 and/or the one or more insulated regions 118. In other words, when energy emission is allowed through active electrode 112, a controlled cutting section may be created by virtue of the structural and/or functional relationship and/or interconnectedness of one or more of the active electrode 112, the one or more cutting regions 116 and/or the one or more insulated regions 118 and/or one or more insulating elements 130. As a non-limiting example, a practitioner and/or user of one or more of the electrosurgical ablation devices disclosed herein may position an electrosurgical ablation device 100 within and/or proximate to a desired area of treatment. A user and/or practitioner may thereafter activate the electrosurgical ablation device 100, and may emit energy from active electrode 112 to cut and/or otherwise ablate tissue as the active electrode 112 passes along and/or about access cannula 114, thereby providing the ability to create a tract, passage, fistula, and/or other dilated region since the access cannula 114 forms a return electrode by virtue of placement of the one or more cutting sections 116; which may be formed by an area of the access cannula 114 devoid of insulating and/or dielectric material.

As a portion of the access cannula 114 is selectively insulated to form the shutoff section 118, cutting can only be completed when the outer sheath 104 is sliding over the non-insulated portion, or the cutting section 116, of the access cannula. For example, cutting (or tissue ablation due to the application of RF energy) will only take place in region 126 and will cease in region 128. That is, when the outer sheath is advanced to region 128, application of energy will cease as active electrode 112 no longer has a short electrical path to return electrode 116 because of the insulated nature of 118.

FIG. 1B illustrates an RF ablation system 100 that includes the electrosurgical ablation device 102. During a procedure (e.g., an HGS procedure as depicted in FIG. 1A, or the like) the access cannula 114 and the electrode 112 may be connected or otherwise coupled to the electrosurgical generator 150 by conductive wires 154 and 152, respectively. During operation, the electrosurgical generator 150 can apply current to the electrode 112 and current will flow between the non-insulated portion of the access cannula 114 and the electrode 112 when in close proximity of one another. More specifically, current will flow between the electrode 112 and cutting section 116 when the cutting section 116 is extended out of the lumen of the outer sheath 104. As such, where the cutting section 116 (or a portion of the cutting section 116) is exposed a complete pathway for current is formed, thereby ablating or otherwise cutting the tissue in the path between 112 and 116. Using the example depicted in FIG. 1A, an anastomosis could be formed between the stomach 120 and the duct 124 to enable treatment (e.g., drainage, placement of a stent, or the like) when the outer sheath electrode 112 is passed over the access cannula 114 with energy applied.

The electrosurgical ablation device 102 of the present disclosure provides significant advantages over conventional devices. For example, as the current device does not require the patient to be grounded, and therefore more fine control over the cutting region (e.g., region 126) can be achieved. The electrosurgical generator 150 can be configured such that current will only flow between the electrode 112 and uninsulated portions of the access cannula 114 (e.g., the cutting section 116) within a preselected distance from the electrode 112. Preselected distance 112 may be a predefined distance, a preset distance, and/or other like configuration of quantified distances. In this and other examples, the preselected distance and/or other aforementioned quantified distances may be set, preset, and/or selected and/or preselected to be set and/or modified to a distance of about 0.005 mm or more. In other non-limiting examples, the preselected distance and/or other aforementioned quantified distances may be set, preset, and/or selected and/or preselected to be set and/or modified to a distance of about 0.05 mm or more. In yet other non-limiting examples, the preselected distance and/or other quantified distances may be set, preset, and/or selected and/or preselected to be set and/or modified to that is any distance within a range of 0.0005 mm to 1 mm or more.

In some examples, the shutoff section 118 of the access cannula 114 may be composed of one or more insulating materials, or two or more insulating materials. In other words, the applied insulating material applied to the shutoff section 118 of the access cannula may be a blend of insulating materials, a mixture of insulating materials, a matrix of insulating materials, a weave of insulating materials, a braid of insulating materials, a pattern of insulating materials, a patterned array of insulating materials, or other like configurations. It can be appreciated, given the spatial relationship of the cutting section 116 to the shutoff section 118 as described above, that the spatial configuration (i.e., surface area) of the cutting section 116 may directly affect the spatial configuration of the shutoff section 118, since the cutting section 116 is formed by any portion or section of the access cannula 114 that is devoid of applied insulating material.

In this and other examples, the shutoff section 118 may include a dielectric material. Alternatively, or additionally, the shutoff section 118 may include one or more dielectric materials. Various configurations and arrays of the shutoff section 118 are further contemplated by the present disclosure and will be further described herein. In some instances, the shutoff section 118 may include applied insulating materials, or one or more applied insulating materials and may form a patterned arrangement along the body of the access cannula 114. Such patterned arrangements may include, but are not limited to intermittent patterns, dotted patterns, raised-patterns (i.e., patterns with a vertical profile and thickness), relief-patterns (i.e., patterns with depth including but not limited to etches and grooves), sinusoidal patterns, helical patterns, serpentine patterns, jigsaw patterns, and/or the like. Alternatively, or additionally, the applied insulating materials may be applied to create a shutoff section 118 in the form of a coating, a covering, and/or a jacket. Alternatively, or additionally, the applied insulating materials may be applied to create a shutoff section 118 in the form of a partial coating, a partial covering, and/or a partial jacket. In yet other non-limiting examples, the applied insulating materials may be applied to create a shutoff section 118 in the form of a substantial coating (i.e., a coating covering at least 80% of the circumference of the access cannula 114), a substantial covering (i.e., a covering encapsulating at least 80% of the circumference of the access cannula 114), and/or a substantial jacket (i.e., a jacket covering at least 80% of the circumference of the access cannula 114).

With some embodiments, multiple access cannulas 114 can be provided for each outer sheath 104 where each access cannula 114 has a different length of cutting section 116. Alternatively, or additionally, the length and/or size and/or surface area of any cutting section disclosed herein may be customized and/or otherwise modified and/or adapted to be sized in accordance with a given subject and/or patient. In other words, the length and/or size and/or surface area of any cutting section disclosed herein may be customized and/or otherwise modified and/or adapted to accurately conform to the desired area of treatment, such that energy and/or therapy may be delivered to the desired area of treatment precisely and accurately, regardless of the size of the desired area of treatment and/or regardless of the size and/or any dimensions of the patient and/or subject.

Accordingly, given the interrelated spatial relationships of the cutting section 116 and the shutoff section 118, the size of each respective section may be customized by the user/practitioner of the electrosurgical ablation device 102 to be tailored to the size of the desired treatment area of a patient or subject. In other words, the size and/or any other dimensional quantity (i.e., surface area, volume, width, height, length, etc.) of both the cutting section 116 and the shutoff section 118 may be selected by selecting an access cannula 114 for use with the outer sheath 104 that cutting section 116 and shutoff section 118 matching a desired sizing to ablate the tissue of the patient. In this manner. both the cutting section 116 and the shutoff section 118 may be tailored for patient-specific anatomy and/or treatment.

Once the electrosurgical ablation device 102 is placed within the desired treatment site, energy may be applied to the desired treatment site through a conductive path formed by the active electrode 112 and the cutting section 116 to locally ablate tissue. Energy may be delivered to the active electrode 112 by the electrosurgical generator 150 through the connection of a lead wire which will be further described herein. Forms of energy applied and/or generated by the electrosurgical generator 150 include but are not limited to RF energy, electrical energy, ultrasonic energy, thermal energy, or any combination or permutation of the aforementioned. Alternatively, or additionally, the access cannula 114 may be translated (i.e., moved) along a proximal and/or distal direction to effectively move the shutoff sections 118 into a shutoff condition, thereby interrupting the current pathway with the active electrode 112 and stopping all energy application from the electrosurgical ablation device 102 to the treatment site. Alternatively, or additionally, the outer sheath 104 may be translated (i.e., moved) over the access cannula 114 along a proximal and/or distal direction to effectively move the shutoff sections into a shutoff condition, thereby interrupting the current pathway with the active electrode 112 and stopping all energy application from the electrosurgical ablation device 102 to the treatment site. Various other configurations of shutoff conditions for the devices disclosed herein are contemplated and will be discussed further herein.

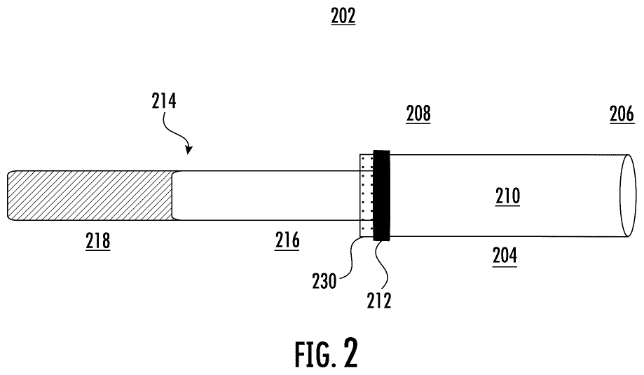

FIG. 2 illustrates a side view of an electrosurgical ablation device in accordance with examples and embodiments of the present disclosure. As shown in FIG. 2, and applicable to other examples, an electrosurgical ablation device 202 may be deployable to a desired treatment site through any of a variety of minimally invasive procedures (e.g., endoscopic access, or the like). In this and other examples, an electrosurgical ablation device 202 may include an outer sheath 204 having a proximal end 206, a distal end 208, and a lumen 210 extending therebetween. Outer sheath 204 may further include an active electrode 212 disposed proximate to the distal end 208 and may further include and/or incorporate an insulating element 230 disposed proximate to the distal end 208, the active electrode 212, and/or both.

The insulating element 230 may be annular (i.e., ring-shaped) and/or conform to various other shapes and/or geometries with the intent to create a set distance between 212 and 216 and prevent shorting of the electrodes against each other. By non-limiting examples, the insulating element 230 of this and other examples may be square-shaped, rectangular, oblong, obloid, ellipsoidal, spherical, diamond-shaped, spiral-shaped, prism-shaped, cuboid, may be a shape including one or more projections, may be a shape including multiple projections, or a shape and/or geometry partially resembling any of the shapes and/or geometries and/or the like. Alternatively, or additionally, the active electrode 212 of this and other examples may be annular (i.e., ring-shaped) and/or conform to various other shapes and/or geometries. By non-limiting examples, the active electrode of this and other examples may be square-shaped, rectangular, oblong, obloid, ellipsoidal, spherical, diamond-shaped, spiral-shaped, prism-shaped, cuboid, may be a shape including one or more projections, may be a shape including multiple projections, or a shape and/or geometry partially resembling any of the shapes and/or geometries and/or the like. Alternatively, or additionally, any of the active electrodes described herein may be cutting electrodes. In other words, any of the active electrodes described herein may include cutting features and/or cutting elements which act to ablate tissue and/or dilate tissue. In yet other non-limiting examples, any of the active electrodes described herein may be adapted for cutting tissue, dilating tissue, and/or ablating tissue.

An access cannula 214 may be slidably disposed within the outer sheath 204 and more particularly may be slidably disposed within a lumen 210 of the outer sheath 204 or one or more lumens disposed within the outer sheath 204. Access cannula 214 may be composed of conductive material and may be composed of one or more or two or more conductive materials. Insulating, insulative, or dielectric material may be applied to the body of the access cannula 214 to form one or more non-conductive shutoff sections 218. The non-conductive shutoff sections 218, due to their insulating and/or non-conductive nature, may be adapted to stop all energy transmission from the electrosurgical ablation device under certain conditions as will be described further herein.

The one or more cutting sections 216 and the active electrode 212 may be coupled to an electrosurgical generator (as shown in FIG. 1B) or other energy delivery device such that the one or more cutting sections 216 and the active electrode 212 may form a conductive path to ablate tissue of a patient or subject that is located between the electrodes.

Alternatively, or additionally, the access cannula 214 may take the form of a guidewire, an obturator, a dilator, a stylet, a pusher tool and/or pusher rod, an elongated device, a probe, a needle, a cystotome, a sphincterotome, or any like device designed to be introduced within a patient and/or subject.

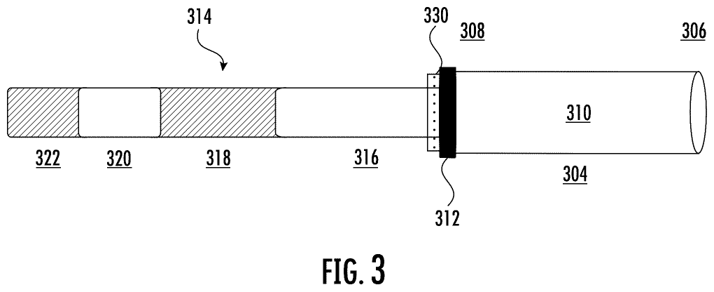

FIG. 3 depicts an electrosurgical ablation device 302 in accordance with examples and embodiments of the present disclosure. As shown in FIG. 3, an electrosurgical ablation device 302 comprises an outer sheath 304 that includes a proximal end 306, a distal end 308, a lumen 310 extending therebetween, and an active electrode 312 disposed proximate to the distal end 308 of the outer sheath 304. Further contemplated and shown in FIG. 3 is an access cannula 314 which may be slidably disposed within the outer sheath 304. The access cannula 314 may have a body composed of an electrically conductive material which may include an insulating material applied to one or more portions of the body to form one or more shutoff sections (318, 322) and one or more cutting sections (316, 320). The one or more cutting sections (316, 320) and the active electrode 312 may be coupled to a generator such that the one or more cutting sections (316, 320) and the active electrode 312 may form a conductive path to ablate tissue of a patient or subject when in proximity of each other.

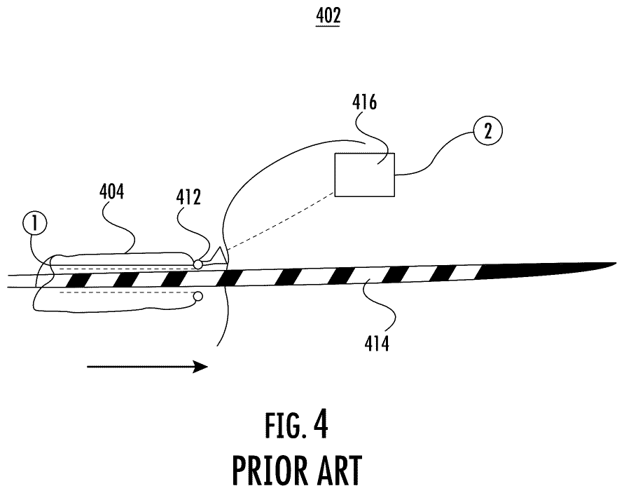

FIG. 4 shows a conventional dilation catheter of the prior art, which utilizes monopolar RF energy, and a grounding pad attached to the patient or subject. As shown, the patient is typically used as the ground through the utilization of a grounding pad or other grounding electrode 416 in electrosurgical ablative procedures carried out by this device. Compared to the present disclosure, the prior art device of FIG. 4 presents several disadvantages that result in less effective treatment, less precise treatment, and increased risk to the patient. For instance, since the patient is typically used as the ground, energy delivered through active electrode 412 will be applied when in contact with any portion of the patient, requiring very precise physical control of the catheter by physician. Further, the prior art device shown in FIG. 4 is incapable of automatically shutting off a current path without first shutting off and/or disabling a connected electrosurgical generator (not shown) or other like device, and/or another device that communicates with the electrosurgical generator, such as a foot pedal or other modulating device. Accordingly, access cannula 414 and/or a guidewire and/or any like introducing element of the prior art device is not composed of conductive material and/or is not a return electrode, ground electrode, or an electrode of any kind since the structure of the prior art device could not allow for this as the prior art device would short circuit. In other words, if the access cannula 414 of the prior art device were conductive, the active electrode 412 would immediately short circuit as the current would take the path of least resistance and likely apply energy at an unintended location in the body, causing harm. The prior art device shown in FIG. 4 may also include an outer sheath 404, however, sliding the outer sheath 404 of the prior art device while the device is in operation would result in a short circuit if the access cannula 414 was composed of conductive material. Thereby belying myriad unobvious advantages of the present disclosure as the devices of the present disclosure overcome at least all the limitations of the prior art device.

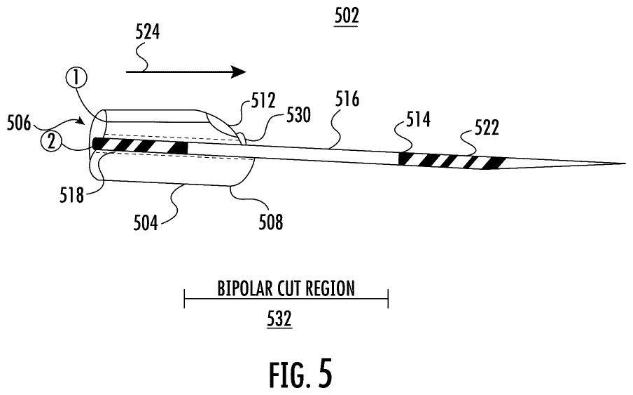

FIG. 5 shows a device of the present disclosure which may include a slidably engaged bipolar electrode system. As shown in FIG. 5, a bipolar cutting region 532 is created by virtue of a conductive path created between active electrode 512 and access cannula 514, which acts as a return and/or ground electrode. Further shown in FIG. 5 are shutoff sections 518 and 522 which may be composed of the same or like material as any of the shutoff sections disclosed herein. The device of this and other examples further includes at least one cutting region 516 but may include only one cutting region 516 in other examples. Cutting region 516 may be composed of the same or like material as any of the cutting regions disclosed herein. Advantageously, outer sheath 504 and access cannula 514 are translatable or otherwise movable with relation to each other and the patient such that the bipolar cut region 532 may be selectively adjusted by the user or practitioner prior to energy activation. In other words, outer sheath 504 may be movable relative to the one or more cutting sections 516 and/or the one or more shutoff sections (518, 522) such that the size of the bipolar cutting region 532 (i.e., length, area, surface area, volume, height, width, etc.) may be adjusted given the circuitous relationship between the components of the devices in all examples as described herein. An insulating element 530 may further be provided to prevent the device from short-circuiting, and to further aid the efficacy in treatment of a patient or subject.

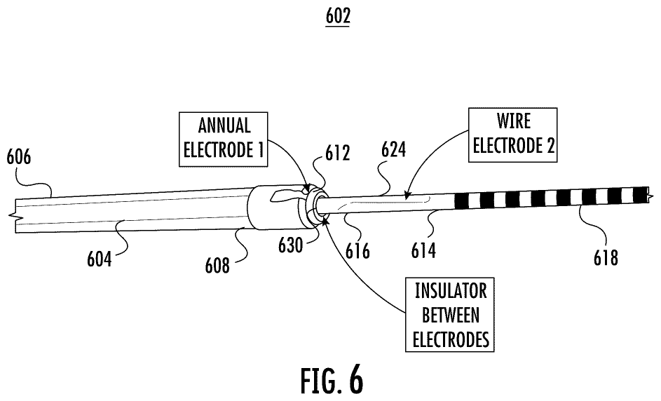

FIG. 6 shows an example electrode configuration for the systems and devices disclosed herein. As shown in FIG. 6, an annular electrode 612 may be provided as an active electrode and a wire electrode 614 may also be provided. Wire electrode 614 may interact with the outer sheath 604, pass through the outer sheath 604, be slidably attached to and/or attached slidably within outer sheath 604, fixedly attached at one or more portions of outer sheath 604, and/or affixed to at least a portion of the outer sheath 604, which includes the outer sheath proximal end 606 and the outer sheath distal end 608. A non-conductive and/or dielectric shutoff section 618 may further be provided which acts to cease all application of energy to the treatment site when the annular electrode 612 passes over the shutoff section 618 and/or reaches a preset distance from the shutoff section 618. Preset distances may be preset anywhere between 0.005 mm to 10 cm or at any distance desired by the user and/or practitioner of the devices disclosed herein. It can be appreciated that preset distances may also be referred to as preselected distances, predefined distances, or by other like and/or known configurations of quantified distances. Alternatively, or additionally, an insulating element 630 may be provided affixed, adhered, bonded and/or otherwise coupled proximate to the annular electrode 612 and/or one or more portions of the annular electrode 612 to prevent short-circuiting of the device and to further aid the efficacy in treatment of a patient or subject. Alternatively, or additionally, wire electrode 614 may be provided in the form of a helically wound wire, a tubular wire, a tube, a shaft, a rod, a guidewire, a tension wire, and may be disposed anywhere within, upon, about, and/or proximate to the annular electrode 612 and/or one or more shutoff sections 618.

Alternatively, or additionally, wire electrode 614 may be disposed about, along, proximate to, and/or otherwise within access cannula 616 and may be disposed about, along, proximate to, or otherwise within access cannula 616 in a slidable, slidably movable, relatively moveable, and/or telescoping relationship and/or nested relationship. In this and other examples, wire electrode 614 may take the form of a tube, a wire, a shaft, a rod, or other similar structure and/or may be hollow, substantially hollow, partially hollow, and/or not hollow (i.e., contiguously and continuously solid in construction). Alternatively or additionally, wire electrode 614 may have a circular cross-section, a substantially circular cross-section, a partially circular cross-section, an elliptical cross-section, a substantially elliptical cross-section, a partially elliptical cross-section, an ovular cross-section, a substantially ovular cross-section, a partially ovular cross-section, a square cross-section, a substantially square cross-section, a partially square cross-section, a rectangular cross-section, a substantially rectangular cross-section, a partially rectangular cross-section and/or a cross section incorporating any combination and/or permutation of the aforementioned cross sections.

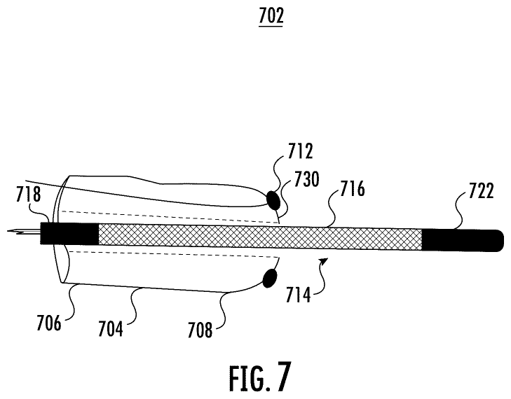

FIG. 7 shows a modified EUS access device with bipolar cutting section. As shown in FIG. 7, and applicable to any or all examples disclosed herein, an outer sheath 704 may be provided in a slidable, coaxial relationship with an access cannula 714. Access cannula 714 may be composed of conductive material and may include one or more conductive cutting sections 716 and one or more sections of applied insulative and/or dielectric material which form one or more non-conductive shutoff sections (718, 722). Further shown in FIG. 7 is another example of an active electrode 712 with an insulative element 730 that may be disposed proximate to the active electrode 712. In this and other examples, energy may be applied to a desired treatment site of a patient or subject through the active electrode 712 and may be controlled, regardless of the operational status of a connected electrosurgical generator or like device, by positioning of the outer sheath 704 relative to the one or more cutting sections 716 and/or the one or more shutoff sections (718, 722). In other words, the user and/or practitioner may selectively control the application region of energy to an exactly defined and desired treatment area by manipulating the active electrode 712 to and from respective cutting sections 716 and/or respective shutoff sections (718, 722) to control energy delivery from the device and/or active electrode 712 of this and other examples independent of the energy being supplied by a connected electrosurgical generator and/or like device. This is since energy is only applied to the desired treatment site when a conductive path is formed when the active electrode 712 and the access cannula 716 are in close proximity, and the fact that energy is no longer applied when the conductive cutting path between the active electrode 712 and the access cannula 716 is interrupted by any shutoff section disclosed herein, such as but not limited to shutoff sections 718 and 722.

Alternatively, or additionally to the examples provided herein, methods of use and methods of treatment incorporating the devices disclosed herein are further contemplated by the present disclosure. An example method incorporating the devices disclosed herein may include positioning an endoscope 140 within a first body lumen of a patient (i.e., an esophagus, GI tract, or the like) adjacent to a desired treatment site (i.e., biliary tract, biliary duct, GI tract, or the like). Methods of this and other examples may further include advancing an electrosurgical ablation device, such as electrosurgical ablation device (102/202/302/502/602/702) through an endoscope, such as endoscope 140, and into the desired area of treatment such that at least one cutting section (116/216/316/320/516/616/716) is placed within the desired area of treatment. Methods of this and other examples may further include activating an electrosurgical generator, such as electrosurgical generator 150, to deliver energy to the electrosurgical ablation device (102/202/302/502/602/702), applying energy to the treatment site through the active electrode (112/212/312/512/612/712) via the electrosurgical generator to locally ablate tissue when the active electrode (112/212/312/512/612/712) passes over the cutting section (116/216/316/320/516/616/716) of the access cannula (114/214/314/514/614/714); and automatically ceasing application of energy to the treatment site through the active electrode (112/212/312/512/612/712) when the active electrode (112/212/312/512/612/712) passes over the one or more shutoff sections (118/218/318/322/518/618/718/722) of the access cannula (114/214/314/514/614/714).

Alternatively, or additionally, methods of the present disclosure may further include wherein the one or more cutting sections (116/216/316/320/516/616/716) include an energy activation region. The energy activation region may be set at a preset distance from the active electrode (112/212/312/512/612/712). In other words, the shutoff sections (118/218/318/322/518/618/718/722) may be arranged at a preset distance from one or both of the active electrode (112/212/312/512/612/712) and/or the one or more cutting sections (116/216/316/320/516/616/716) such that an energy activation region is created when the current path between the active electrode (112/212/312/512/612/712) and the one or more cutting sections (116/216/316/320/516/616/716) is uninterrupted. Accordingly, the electrosurgical ablation device (102/202/302/502/602/702) of this and other examples may be configured to stop applying energy to a treatment site or the vicinity of a treatment site when one or both active electrode (112/212/312/512/612/712) and/or the access cannula (114/214/314/514/614/714) are not within a boundary of the energy activation region. Therefore, the energy activation region of this and other examples may be set to a preset distance. The preset distance to the energy activation region may be the distance from a boundary of the energy activation region (i.e., outer bound) to one or both active electrode (112/212/312/512/612/712) and/or the access cannula (114/214/314/514/614/714). In this and other examples, the preset distance may range from about 0.005 mm to about 5 mm. In other non-limiting examples, the preset distance may range from about 0.0025 mm to about 15 mm. In yet other non-limiting examples, the preset distance may range from about 0.0015 mm to about 50 mm. In further non-limiting examples, the preset distance may be selected by the user and/or practitioner of the device, and the user and/or practitioner of the device may provision the preset distance by the selective application of applied insulating, insulative or dielectric material to the body of the access cannula (114/214/314/514/614/714).

Suitable insulating and/or insulative materials that may be applied to the body of the access cannula (114/214/314/514/614/714) may include but are not limited to: polystyrene, polyethylene, porous polyethylene, polyether ether ketone (PEEK), ceramic, Teflon, fiberglass, cellulose, elastomeric materials, rubbers, plastics, thermoplastics (i.e., temperature-dependent plastics), polymers, synthetic polymers, fluoropolymers, thermopolymers (i.e., temperature-dependent polymers), or other like insulating and/or insulative materials. Alternatively, or additionally, any of the materials may be applied via a spray, a coating, a jacket, an extrusion process, a reflow process, a stamping process, a branding process, a bonding process, a thermal process, and/or other like process(es) known in the art.

Suitable dielectric materials that may be applied to the body of the access cannula (114/214/314/514/614/714) may include but are not limited to: ceramics, glass, plastics, mica, gas dielectrics (e.g., nitrogen, helium), air dielectrics (e.g., atmospheric air), fluid dielectrics (e.g., distilled water), or other like or known dielectrics. Alternatively, or additionally, any of the dielectric materials may be applied via a spray, a coating, a jacket, an extrusion process, a reflow process, a stamping process, a branding process, a bonding process, a thermal process, and/or other like process(es) known in the art.

The access cannula (114/214/314/514/614/714) of any example or embodiment described herein may be composed or comprised of one or more conductive materials, including but not limited to: metals (e.g., copper, gold, tungsten, silver), alloys (e.g., titanium, stainless steel, brass), semi-conductors (e.g., germanium, silicon, selenium), or other like or known materials in the art. Alternatively or additionally, any of the aforementioned conductive materials may be applied via a spray, a coating, a jacket, an extrusion process, a reflow process, a stamping process, a branding process, a bonding process, a thermal process, and/or other like process(es) known in the art.

The active electrode (112/212/312/512/612/712) of any example or embodiment described herein may be composed or comprised of one or more conductive materials, including but not limited to: metals (e.g., copper, gold, tungsten, silver), alloys (e.g., titanium, stainless steel, brass), semi-conductors (e.g., germanium, silicon, selenium), or other like or known materials in the art. Alternatively or additionally, any of the aforementioned conductive materials may be applied via a spray, a coating, a jacket, an extrusion process, a sputtering process, a reflow process, a stamping process, a wire bending process, a branding process, a bonding process, a thermal process, and/or other like process(es) known in the art.

Alternatively, or additionally, multiple active electrodes (112/212/312/512/612/712) may be disposed proximate to the distal end (108/208/308/508/608/708) of the outer sheath (104/204/304/504/604/704) of the electrosurgical ablation device (102/202/302/502/602/702). In other words, and by non-limiting examples, the electrosurgical ablation device may include one or more active electrodes disposed proximate to the distal end of the outer sheath, two or more active electrodes disposed proximate to the distal end of the outer sheath, or three or more active electrodes disposed proximate to the distal end of the outer sheath.

Alternatively, or additionally, the active electrodes (112/212/312/512/612/712) of this and other examples may be provided in an array. In other words, the electrosurgical ablation device (102/202/302/502/602/702) of this and other examples may include an array of active electrodes disposed proximate to the distal end of the outer sheath. In yet other non-limiting examples, the electrosurgical device of this and other examples may include two or more arrays of active electrodes disposed proximate to the distal end of the outer sheath.

Alternatively, or additionally, one or more active electrodes may be disposed remotely from one or more active electrodes (112/212/312/512/612/712) described herein. In other words, one or more active electrodes may be present along, within, attached to or otherwise coupled to any component of electrosurgical ablation device (102/202/302/502/602/702) remote from one or more active electrodes (112/212/312/512/612/712) disposed proximate to the distal end (108/208/308/508/608/708) of the outer sheath (104/204/304/504/604/704), and/or remote from one or more active electrodes disposed anywhere along, within, attached to or otherwise coupled to any component of electrosurgical ablation device (102/202/302/502/602/702). Any of the one or more active electrodes disposed remotely may be disposed at a constant distance, or at varying distances from one or more active electrodes. For instance, and by non-limiting example, any active electrodes may be disposed remote from any other active electrodes by any distance ranging between 0.15 mm and 100 mm.

The active electrode (112/212/312/512/612/712) of any example or embodiment described herein may be composed or comprised of one or more conductive materials, and one or more insulating and/or insulative materials, including but not limited to the conductive materials and the insulating, insulative and/or dielectric materials described herein.

Alternatively, or additionally to the examples described herein, the outer sheath may be coaxially and/or concentrically disposed over the access cannula. The access cannula may include one or more conductive cutting sections and one or more non-conductive shutoff sections. Although not explicitly shown, a tension wire or similar device (e.g., guidewire) may be deployed to initially penetrate through tissue prior to performing a cutting or ablative procedure. The tension wire or other similar device, for example, may be utilized to initially penetrate selected or predetermined luminal walls. A stent or other luminal patency device may be deployed to the treated area after completion of the disclosed procedure, or any procedure carried out with the devices and/or systems of the present disclosure.

Alternatively, or additionally, the access cannula (114/214/314/514/614/714) of the present disclosure may include one or more cutting sections (116/216/316/320/516/616/716), two or more cutting sections, three or more cutting sections, four or more cutting sections, or five or more cutting sections. In other non-limiting examples, the access cannula (114/214/314/514/614/714) of the present disclosure may include one or more shutoff sections (118/218/318/322/518/618/718/722), two or more shutoff sections, three or more shutoff sections, four or more shutoff sections, or five or more shutoff sections.

Alternatively, or additionally, the access cannula (114/214/314/514/614/714) of the present disclosure may include a tip member. A tip member may take the form of an obturator tip, a dilator tip, a cutting tip, a wedge tip, a blunt tip, a rounded tip, an edged tip, a triangular tip, a prismatic tip, a rectangular tip, a flexible tip, a rigid tip, a sharpened tip, a composite tip or any of the like and/or any combination and/or permutation of the aforementioned. A tip member may be molded onto and/or disposed proximate to the distal end of the access cannula (114/214/314/514/614/714) or may be combined with the access cannula (114/214/314/514/614/714) as a unitary part (i.e. of one-piece construction), and/or alternatively may be combined with the access cannula (114/214/314/514/614/714) as a two-piece or two-part construction, as a three-piece or three-part construction, or may combine and/or otherwise couple with the access cannula (114/214/314/514/614/714) in a construction and/or coupling requiring four or more pieces and/or parts and/or components.

The devices of the present disclosure may be delivered via an endoscope and methods of using the devices of the present disclosure may incorporate an endoscope, such as the endoscope as illustrated in FIG. 1A. The devices of the present disclosure may be introduced through the working channel of an endoscope that includes a proximal handle that provides control of delivery and a catheter that extends from the distal end of the proximal handle to the distal end of the device. The handle may have a coupling member for removable attachment to the endoscope to provide a stable deployable platform.

It should be noted and can be appreciated that some of the FIGS. are schematic in nature and are not drawn to scale. Certain features are shown larger than their scale and certain features are omitted from some views for ease of illustration.

It should also be noted that, as used in this specification and the appended claims, the singular forms include the plural unless the context clearly dictates otherwise. It should also be noted that the term “or” is generally employed in its sense including “and/or” unless the content clearly dictates otherwise.

The present disclosure has been described with reference to various specific and exemplary embodiments. Those skilled in the art will understand that changes may be made in detail, particularly in matters of shape, size, material and arrangement of parts. Accordingly, various modifications and changes may be made to the examples and the embodiments. Additional or fewer components may be used, depending on the condition that is being treated by the electrosurgical ablation device and other related devices and components disclosed herein. Many variations and modifications may be made while remaining within the spirit and scope of the present disclosure. The specifications and drawings are, therefore, to be regarded in an illustrative rather than a restrictive sense.

What is claimed is:

1. A radiofrequency (RF) ablation device for treating patient tissue comprising:
an outer sheath;
an access cannula configured to be slidably disposed within the outer sheath;
wherein the outer sheath includes a proximal end, a distal end, a lumen extending therebetween, and an active electrode disposed at or near the outer sheath distal end, the active electrode operatively connected to an RF generator by a lead wire; the outer sheath configured to slidably translate relative to the access cannula;
wherein the access cannula comprises a body composed of an electrically conductive material and is connected to the RF generator by a return wire, thereby forming a ground electrode;
wherein the access cannula further comprises one or more sections of applied insulating material, the one or more sections of applied insulating material forming one or more non-conductive shutoff sections and the remaining sections forming one or more conductive cutting sections;
wherein the one or more conductive cutting sections form a conductive path with the active electrode when the active electrode radially aligns with any portion of the one or more conductive cutting sections;
whereby when RF energy is applied to the active electrode, RF energy passes through the conductive cutting section to selectively ablate tissue at a treatment site only when a conductive path is formed.

2. The device of claim 1, wherein the access cannula is one of a guidewire, a wire, a tube or a shaft.

3. The device of claim 1, wherein the active electrode is one of a ring electrode, annular electrode, cutting ring electrode, or cutting annular electrode.

4. The device of claim 1, wherein the outer sheath and the access cannula are slidable and/or translatable relative to one another.

5. The device of claim 1, wherein the outer sheath further comprises a distal insulating tip member disposed adjacent to the active electrode.

6. The device of claim 1, wherein the applied insulating material is in the form of a jacket, a coating, or a covering applied to the body of the access cannula.

7. The device of claim 1, wherein the outer sheath further comprises a distal insulating tip member bonded or directly coupled to the active electrode.

8. The device of claim 5, wherein the insulating tip member is formed of ceramic.

9. The device of claim 1, wherein the applied insulating material is a dielectric material.

10. The device of claim 1, wherein the conductive path comprises a set conductance distance, wherein the set conductance distance remains constant throughout application of RF energy along the conductive cutting sections.

11. The device of claim 1, wherein the RF ablation device automatically stops applying RF energy to the treatment site when the active electrode radially aligns with any portion of the one or more non-conductive shutoff sections.

12. A radiofrequency (RF) ablation device comprising:
an RF generator;
an access cannula operatively connected to the RF generator by a return wire;
an outer sheath comprising a proximal end, a distal end, and a lumen therebetween;
wherein the outer sheath further comprises an active electrode disposed at or near the distal end, connected to the RF generator by a lead wire;
wherein the access cannula comprises one or more shutoff sections composed of insulating material, and one or more cutting sections composed of conductive material, and is slidably disposed within the lumen of the outer sheath.

13. The device of claim 12, wherein the access cannula is one of a guidewire, a wire, a tube or a shaft.

14. The device of claim 12, wherein the conductive path comprises a set conductance distance, wherein the set conductance distance remains constant throughout application of RF energy along the conductive cutting sections.

15. The device of claim 12, wherein the active electrode applies RF energy along the one or more cutting sections during operation of the RF generator when the active electrode passes along at least one cutting section and is grounded when the active electrode passes along at least one non-conductive shutoff section and applies no RF energy to the one or more cutting sections.

16. A method of treating patient tissue, the method comprising:
positioning an endoscope within a first body lumen of a patient adjacent to a desired treatment site;
advancing a radiofrequency energy (RF) delivery device through the endoscope;
wherein the RF delivery device comprises:
an outer sheath; the outer sheath including a proximal end, a distal end, an active electrode disposed at or near the distal end, and a lumen extending therebetween;
an access cannula slidably disposed within the lumen of the outer sheath; the access cannula including a non-conductive shutoff section and a conductive cutting section;
wherein the RF delivery device is operatively connected to an RF generator;
positioning the RF delivery device at or near a desired treatment site in a treatment configuration;
applying RF energy, from the RF generator through the RF delivery device and to the active electrode;
wherein applying RF energy from the RF generator through the delivery device comprises only delivering RF energy from the active electrode to the conductive cutting section of the access cannula when the active electrode eclipses at least a portion of conductive cutting section of the access cannula.

17. The method of claim 16, wherein the non-conductive shutoff section is formed by applying an insulating material to the access cannula.

18. The method of claim 16, wherein the non-conductive shutoff section is formed by applying a dielectric material to the access cannula.

19. The method of claim 16, wherein the conductive cutting section is sized in relation to the size of the patient.

20. The method of claim 16, wherein the non-conductive shutoff section is sized in relation to the size of the patient.
